(19) United States
(12) Patent Application Publication
(10) Pub. No.: US 2011/0105543 A1
(43) Pub. Date: May 5, 2011

(54) NOVEL TRIARYL DERIVATIVES USEFUL AS MODULATORS OF NICOTINIC ACETYLCHOLINE RECEPTORS

(75) Inventors: Antonio Nardi, Herzogenrath (DE); Jeppe Kejser Christensen, Kobenavn (DK); Dan Peters, Malmo (SE)

(73) Assignee: NeuroSearch A/S, Ballerup (DK)

(21) Appl. No.: 12/922,007

(22) PCT Filed: Mar. 9, 2009
(86) PCT No.: PCT/EP09/52733

§ 371 Date: Nov. 22, 2010

Related U.S. Application Data
(60) Provisional application No. 61/035,915, filed on Mar. 12, 2008.

(30) Foreign Application Priority Data
Mar. 11, 2008 (DK) PA200800371

(51) Int. Cl. A61K 31/505; C07D 239/42; C07C 217/80; C07C 215/74; C07D 403/10; C07D 239/30; C07D 405/10; C07D 239/48; C07D 237/20; C07D 213/73; C07D 401/10; C07D 209/08; C07C 309/01; A61K 31/136; A61K 31/506; A61K 31/50; A61K 31/44; A61K 31/4439; A61K 31/404; A61K 31/185; A61P 25/22; A61P 25/28; A61P 25/00; A61P 25/16; A61P 25/18; A61P 25/24; A61P 3/00; A61P 3/04; A61P 25/20; A61P 25/08; A61P 15/00; A61P 9/12; A61P 9/06; A61P 43/00; A61P 1/12; A61P 5/00; A61P 25/04; A61P 25/06; A61P 29/00; A61P 17/00; A61P 25/30; A61P 25/36; A61P 25/34

(52) U.S. Cl. 514/275; 544/322; 564/307; 544/332; 544/224; 546/311; 546/277.4; 548/503; 562/37; 514/256; 514/647; 514/247; 514/352; 514/339; 514/415; 514/576

(57) ABSTRACT

This invention relates to novel triaryl derivatives, formula (I), a stereoisomer thereof or a mixture of its stereoisomers, or a pharmaceutically acceptable salt thereof, wherein all of X, Y and Z represent CH; or one or two of X, Y and Z represent N; and the others of X, Y and Z represent CH; and R¹, R², R³, R⁴ and R⁵. independently of each other, represent hydrogen, halo, tri-fluoromethyl, trifluoromethoxy, cyano, hydroxyl, alkoxy, alkyl, amino or sulfamoyl; or R¹ and R², together with the phenyl ring to which they are attached form an indolyl ring or a benzo-dioxolyl ring; and R³, R⁴ and R⁵ are as defined above; and R⁶ represents amino or nitro, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

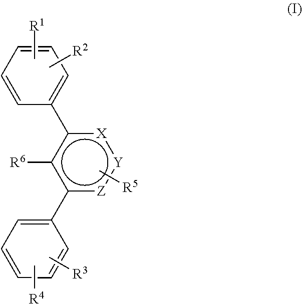

### TECHNICAL FIELD

This invention relates to novel triaryl derivatives derivatives, which are found to be modulators of the nicotinic acetylcholine receptors. Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

### BACKGROUND ART

The endogenous cholinergic neurotransmitter, acetylcholine, exert its biological effect via two types of cholinergic receptors, the muscarinic Acetyl Choline Receptors (mAChR) and the nicotinic Acetyl Choline Receptors (nAChR).

As it is well established that muscarinic acetylcholine receptors dominate quantitatively over nicotinic acetylcholine receptors in the brain area important to memory and cognition, and much research aimed at the development of agents for the treatment of memory related disorders have focused on the synthesis of muscarinic acetylcholine receptor modulators.

Recently, however, an interest in the development of nAChR modulators has emerged. Several diseases are associated with degeneration of the cholinergic system i.e. senile dementia of the Alzheimer type, vascular dementia and cognitive impairment due to the organic brain damage disease related directly to alcoholism.

In the literature, triphenyl-amines are known from various studies of polyphenyls and used as intermediate compounds for the synthesis of olefins, see e.g.

Meinhard D, Wegner M, Kipiani G, Hearley A, Reuter P, Fischer S, Marti O, Rieger B: New Nickel(II) Diimine Complexes and the Control of Polyethylene Microstructure by Catalyst Design; Journal of the American Chemical Society 2007 129 (29) 9182-9191;

Miura Y, Momoki M, Nakatsuji M, Teki Y: Stable thioaminyl radicals having functional groups: generation, ESR spectra, isolation, x-ray crystallographic analyses, and magnetic characterization of N-(arylthio)-4-(ethoxycarbonyl)-2,6-diarylphenylaminyls, N-(arylthio)-4-acetyl-2,6-diarylphenylaminyls, and N-(arylthio)-4-cyano-2,6-diarylphenylaminyls; Journal of Organic Chemistry 1998 63 (5) 1555-1565;

Miura Y, Kurokawa S, Nakatsuji M, Ando K, Teki Y: Pyridyl-Substituted Thioaminyl Stable Free Radicals: Isolation, ESR Spectra, and Magnetic Characterization; Journal of Organic Chemistry 1998 63 (23) 8295-8303;

Miura Y, Nishi T, Teki Y: Isolation and Magnetic Properties of Heterocycle-Carrying N-Alkoxyarylaminyl Radicals; Journal of Organic Chemistry 2003 68 (26) 10158-10161;

Dubovenko Z D, Mamaev V P: Pyrimidines. 72. Synthesis and some properties of 5-amino-2-R-4,6-diphenylpyrimidines and their reaction products; Khimiya Geterotsiklicheskikh Soedinenii 1980 (9) 1278-1282; and

Osisanya R A, Oluwadiya J O: Synthesis of N-heterocycles via chalcone epoxides. 1. Amino- and hydrazinopyrimidines; Journal of Heterocyclic Chemistry 1989 26 (4) 947-948.

However, triaryl derivatives of the present invention are not reported and their activity as modulators of the nicotinic receptors never suggested.

### SUMMARY OF THE INVENTION

The present invention is devoted to the provision novel modulators of the nicotinic receptors, which modulators are useful for the treatment of diseases or disorders related to the cholinergic receptors, and in particular the nicotinic acetylcholine α7 receptor subtype.

The compounds of the invention may also be useful as diagnostic tools or monitoring agents in various diagnostic methods, and in particular for in vivo receptor imaging (neuroimaging), and they may be used in labelled or unlabelled form.

In its first aspect the invention provides triaryl derivatives of Formula I

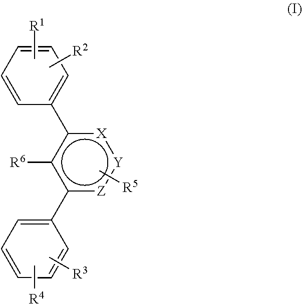

a stereoisomer thereof or a mixture of its stereoisomers, or a pharmaceutically acceptable salt thereof, wherein

all of X, Y and Z represent CH; or one or two of X, Y and Z represent N, and the others of X, Y and Z represent CH; and

R¹, R², R³, R⁴ and R⁵, independently of each other, represent hydrogen, halo, trifluoromethyl, trifluoromethoxy, cyano, hydroxyl, alkoxy, alkyl, amino or sulfamoyl; or R¹ and R², together with the phenyl ring to which they are attached form an indolyl ring or a benzo-dioxolyl ring; and R³, R⁴ and R⁵ are as defined above; and

R⁶ represents amino or nitro;

provided, however,

that not all of R¹, R², R³, R⁴ and R⁵ represent hydrogen;

if R⁵ represents halo, methoxy or amino, not all of R¹, R², R³ and R⁴ represent hydrogen;

if all of X, Y and Z represent CH, then one of R¹ and R², or one of R³ and R⁴, do not represent chloro if the other two of R¹, R², R³ and R⁴ represent hydrogen; and

if X and Z represent N, and R⁵ represent hydrogen, then one of R¹ and R², or one of R³ and R⁴, do not represent methoxy if the other two of R¹, R², R³ and R⁴ represent hydrogen.

In a second aspect the invention provides pharmaceutical compositions comprising a therapeutically effective amount of the triaryl derivative of the invention, or a pharmaceutically acceptable addition salt thereof, together with at least one pharmaceutically acceptable carrier or diluent.

Viewed from another aspect the invention relates to the use of the triaryl derivative of the invention, or a pharmaceutically acceptable addition salt thereof, for the manufacture of pharmaceutical compositions/medicaments for the treatment, prevention or alleviation of a disease or a disorder or a condition of a mammal, including a human, which disease, disorder or condition is responsive to modulation of cholinergic receptors.

In yet another aspect the invention provides a method for treatment, prevention or alleviation of diseases, disorders or conditions of a living animal body, including a human, which disorder, disease or condition is responsive to modulation of cholinergic receptors, and which method comprises the step of administering to such a living animal body in need thereof a therapeutically effective amount of the triaryl derivative of the invention.

Other objects of the invention will be apparent to the person skilled in the art from the following detailed description and examples.

### DETAILED DISCLOSURE OF THE INVENTION

### Triaryl Derivatives

In its first aspect the invention provides triaryl derivatives of Formula I

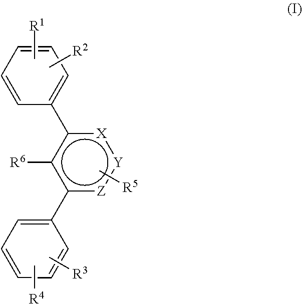

a stereoisomer thereof or a mixture of its stereoisomers, or a pharmaceutically acceptable salt thereof, wherein

all of X, Y and Z represent CH; or one or two of X, Y and Z represent N; and the others of X, Y and Z represent CH; and

R¹, R², R³, R⁴ and R⁵, independently of each other, represent hydrogen, halo, trifluoromethyl, trifluoromethoxy, cyano, hydroxyl, alkoxy, alkyl, amino or sulfamoyl; or R¹ and R², together with the phenyl ring to which they are attached form an indolyl ring or a benzo-dioxolyl ring; and R³, R⁴ and R⁵ are as defined above; and

R⁶ represents amino or nitro;

provided, however,

that not all of R¹, R², R³, R⁴ and R⁵ represent hydrogen (i.e. at least one of R¹, R², R³, R⁴ and R⁵ is different from hydrogen);

if R⁵ represents halo, methoxy or amino, not all of R¹, R², R³ and R⁴ represent hydrogen;

if all of X, Y and Z represent CH, then one of R¹ and R², or one of R³ and R⁴, do not represent chloro if the other two of R¹, R², R³ and R⁴ represent hydrogen; and

if X and Z represent N, and R⁵ represent hydrogen, then one of R¹ and R², or one of R³ and R⁴, do not represent methoxy if the other two of R¹, R², R³ and R⁴ represent hydrogen.

In a preferred embodiment the triaryl derivative of the invention is a compound represented by Formula IA

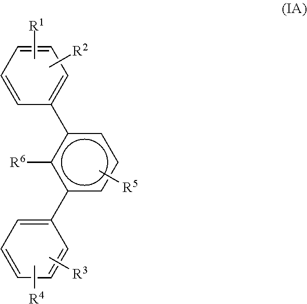

a stereoisomer thereof or a mixture of its stereoisomers, or a pharmaceutically acceptable salt thereof, wherein R¹, R², R³, R⁴, R⁵ and R⁶ are as defined above.

In another preferred embodiment the triaryl derivative of the invention is a compound represented by Formula IB

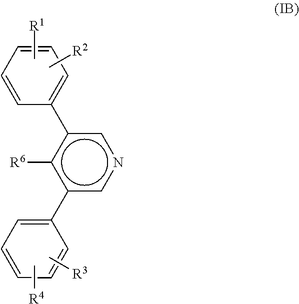

a stereoisomer thereof or a mixture of its stereoisomers, or a pharmaceutically acceptable salt thereof, wherein R¹, R², R³, R⁴ and R⁶ are as defined above.

In a third preferred embodiment the triaryl derivative of the invention is a compound represented by Formula IC

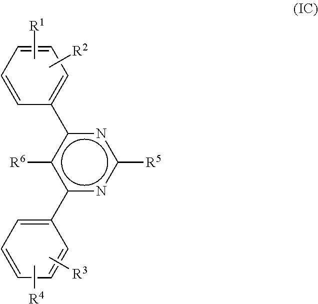

a stereoisomer thereof or a mixture of its stereoisomers, or a pharmaceutically acceptable salt thereof, wherein R¹, R², R³, R⁴, R⁵ and R⁶ are as defined above.

In a fourth preferred embodiment the triaryl derivative of the invention is a compound represented by Formula ID

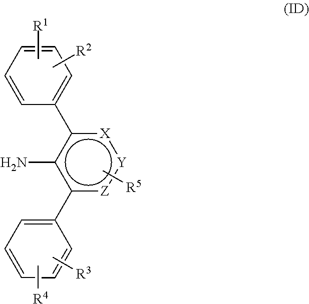

a stereoisomer thereof or a mixture of its stereoisomers, or a pharmaceutically acceptable salt thereof, wherein

all of X, Y and Z represent CH; or one or two of X, Y and Z represent N; and the others of X, Y and Z represent CH; and

R¹, R², R³, R⁴ and R⁵, independently of each other, represent hydrogen, halo, trifluoromethyl, trifluoromethoxy, cyano, hydroxyl or alkoxy.

In a more preferred embodiment the triaryl derivative of the invention is a compound represented by Formula I or ID, wherein all of X, Y and Z represent CH.

In another more preferred embodiment the triaryl derivative of the invention is a compound represented by Formula I or ID, wherein one or two of X, Y and Z represent N; and the others of X, Y and Z represent CH.

In a third more preferred embodiment the triaryl derivative of the invention is a compound represented by Formula I or ID, wherein one of X, Y and Z represent N; and the remaining two of X, Y and Z represent CH.

In a fourth more preferred embodiment the triaryl derivative of the invention is a compound represented by Formula I or ID, wherein Y represents N; and X and Z represent CH.

In a fifth more preferred embodiment the triaryl derivative of the invention is a compound represented by Formula I or ID, wherein two of X, Y and Z represent N; and the remaining one of X, Y and Z represents CH.

In a sixth more preferred embodiment the triaryl derivative of the invention is a compound represented by Formula I or ID, wherein X represents CH; and Y and Z represent N.

In a seventh more preferred embodiment the triaryl derivative of the invention is a compound represented by Formula I or ID, wherein X and Z represent N; and Y represents CH.

In a fifth preferred embodiment the triaryl derivative of the invention is a compound represented by Formula I, IA, IB or IC, a stereoisomer thereof or a mixture of its stereoisomers, or a pharmaceutically acceptable salt thereof, wherein R¹, R², R³, R⁴ and R⁵, independently of each other, represent hydrogen, halo, trifluoromethyl, trifluoromethoxy, cyano, hydroxyl, alkoxy, alkyl, amino or sulfamoyl; or R¹ and R², together with the phenyl ring to which they are attached form an indolyl ring or a benzo-dioxolyl ring; and R³, R⁴ and R⁵ are as defined above; and R⁶ represents amino or nitro; or a compound represented by Formula ID wherein R¹, R², R³, R⁴ and R⁵ are as defined here.

In a more preferred embodiment the triaryl derivative of the invention is a compound represented by Formula I, IA, IB, IC or ID, wherein R¹, R², R³, R⁴ and R⁵, independently of each other, represent hydrogen, halo, trifluoromethyl, trifluoromethoxy, cyano, hydroxyl, alkoxy, alkyl, amino or sulfamoyl.

In another more preferred embodiment the triaryl derivative of the invention is a compound represented by Formula I, IA, IB, IC or ID, wherein R¹, R², R³, R⁴ and R⁵, independently of each other, represent hydrogen, halo, trifluoromethyl, trifluoromethoxy, cyano, hydroxyl or alkoxy.

In a third more preferred embodiment the triaryl derivative of the invention is a compound represented by Formula I, IA, IB, IC or ID, wherein R¹, R², R³, R⁴ and R⁵, independently of each other, represent hydrogen, halo, trifluoromethyl, hydroxyl or alkoxy.

In a sixth preferred embodiment the triaryl derivative of the invention is a compound represented by Formula I, IA, IB, IC or ID, a stereoisomer thereof or a mixture of its stereoisomers, or a pharmaceutically acceptable salt thereof, wherein R¹ represents hydrogen, halo, and in particular fluoro, hydroxy or alkoxy, and in particular methoxy; and R² represents hydroxy, alkoxy, and in particular methoxy, or sulfamoyl.

In a more preferred embodiment the triaryl derivative of the invention is a compound represented by Formula I, IA, IB, IC or ID, wherein R¹ represents hydrogen; and R² represents hydroxy, alkoxy, and in particular methoxy, or sulfamoyl.

In another more preferred embodiment the triaryl derivative of the invention is a compound represented by Formula I, IA, IB, IC or ID, wherein R¹ represents halo, and in particular chloro, hydroxy or alkoxy, and in particular methoxy; and R² represents hydroxy or alkoxy, and in particular methoxy.

In a third more preferred embodiment the triaryl derivative of the invention is a compound represented by Formula I, IA, IB, IC or ID, wherein R¹ represents hydroxy or alkoxy, and in particular methoxy; and R² represents hydroxy or alkoxy, and in particular methoxy.

In a fourth more preferred embodiment the triaryl derivative of the invention is a compound represented by Formula I, IA, IB, IC or ID, wherein R¹ represents halo, chloro; and R² represents hydroxy or alkoxy, and in particular methoxy.

In a fifth more preferred embodiment the triaryl derivative of the invention is a compound represented by Formula I, IA, IB, IC or ID, wherein R¹ represents hydrogen; and R² represents hydroxy or alkoxy, and in particular methoxy.

In a seventh preferred embodiment the triaryl derivative of the invention is a compound represented by Formula I, IA, IB, IC or ID, a stereoisomer thereof or a mixture of its stereoisomers, or a pharmaceutically acceptable salt thereof, wherein R¹ and R², together with the phenyl ring to which they are attached form an indolyl ring or a benzo-dioxolyl ring.

In a more preferred embodiment the triaryl derivative of the invention is a compound represented by Formula I, IA, IB, IC or ID, wherein R¹ and R², together with the phenyl ring to which they are attached form an indolyl ring, and in particular 1H-indol-5-yl.

In another more preferred embodiment the triaryl derivative of the invention is a compound represented by Formula I, IA, IB, IC or ID, wherein R¹ and R², together with the phenyl ring to which they are attached form a benzo-dioxolyl ring, and in particular benzo[1,3]dioxol-5-yl.

In an eight preferred embodiment the triaryl derivative of the invention is a compound represented by Formula I, IA, IB, IC or ID, a stereoisomer thereof or a mixture of its stereoisomers, or a pharmaceutically acceptable salt thereof, wherein R³ and R⁴, independently of each other, represent halo, trifluoromethyl, trifluoromethoxy or cyano.

In a more preferred embodiment the triaryl derivative of the invention is a compound represented by Formula I, IA, IB, IC or ID, wherein R³ and R⁴, independently of each other, represent halo or trifluoromethyl.

In another more preferred embodiment the triaryl derivative of the invention is a compound represented by Formula I, IA, IB, IC or ID, wherein R³ represents halo, and in particular fluoro or chloro; and R⁴ represents trifluoromethyl.

In a ninth preferred embodiment the triaryl derivative of the invention is a compound represented by Formula I, IA, IB, IC or ID, a stereoisomer thereof or a mixture of its stereoisomers, or a pharmaceutically acceptable salt thereof, wherein R⁵ represents hydrogen, halo, and in particular fluoro, trifluoromethyl alkyl, and in particular methyl, or amino.

In a more preferred embodiment the triaryl derivative of the invention is a compound represented by Formula I, IA, IB, IC or ID, wherein R⁵ represents hydrogen, halo, and in particular fluoro, or trifluoromethyl.

In another more preferred embodiment the triaryl derivative of the invention is a compound represented by Formula I, IA, IB, IC or ID, wherein R⁵ represents hydrogen, halo, and in particular fluoro, alkyl, and in particular methyl, or amino. In a third more preferred embodiment the triaryl derivative of the invention is a compound represented by Formula I, IA, IB, IC or ID, a stereoisomer thereof or a mixture of its stereoisomers, or a pharmaceutically acceptable salt thereof, wherein R⁵ represents hydrogen or halo, and in particular fluoro.

In a fourth more preferred embodiment the triaryl derivative of the invention is a compound represented by Formula I, IA, IB, IC or ID, wherein R⁵ represents hydrogen, fluoro, methyl or amino.

In a fifth more preferred embodiment the triaryl derivative of the invention is a compound represented by Formula I, IA, IB, IC or ID, wherein R⁵ represents hydrogen.

In a sixth more preferred embodiment the triaryl derivative of the invention is a compound represented by Formula I, IA, IB, IC or ID, wherein R⁵ represents halo, and in particular fluoro.

In a seventh more preferred embodiment the triaryl derivative of the invention is a compound represented by Formula I, IA, IB, IC or ID, wherein R⁵ represents alkyl, and in particular methyl.

In an eight more preferred embodiment the triaryl derivative of the invention is a compound represented by Formula I, IA, IB, IC or ID, wherein R⁵ represents amino.

In a tenth preferred embodiment the triaryl derivative of the invention is a compound represented by Formula I, IA, IB or IC, a stereoisomer thereof or a mixture of its stereoisomers, or a pharmaceutically acceptable salt thereof, wherein R⁶ represents amino or nitro.

In an eleventh preferred embodiment the triaryl derivative of the invention is a compound represented by Formula I, IA, IB, IC or ID, a stereoisomer thereof or a mixture of its stereoisomers, or a pharmaceutically acceptable salt thereof, wherein

R¹ represents hydrogen;

R² represents hydroxy or alkoxy, and in particular methoxy; and

one of R³ and R⁴ represents halo, and in particular fluoro; and the other of R³ and R⁴ represents trifluoromethyl, trifluoromethoxy or cyano; or both of R³ and R⁴ represent halo, trifluoromethyl, trifluoromethoxy or cyano.

In a more preferred embodiment the triaryl derivative of the invention is a compound represented by Formula I, IA, IB, IC or ID, wherein

R¹ represents hydrogen;

R² represents hydroxy or alkoxy, and in particular methoxy; and

one of R³ and R⁴ represents halo, and in particular fluoro; and the other of R³ and R⁴ represents trifluoromethyl, trifluoromethoxy or cyano; or both of R³ and R⁴ represent halo, trifluoromethyl, trifluoromethoxy or cyano; and

R⁵ represents hydrogen.

In another more preferred embodiment the triaryl derivative of the invention is a compound represented by Formula I, IA, IB, IC or ID, wherein

R¹ represents hydrogen;

R² represents sulfamoyl; and

one of R³ and R⁴ represents halo, and in particular fluoro; and the other of R³ and R⁴ represents trifluoromethyl, trifluoromethoxy or cyano; or both of R³ and R⁴ represent halo, trifluoromethyl, trifluoromethoxy or cyano; and

R⁵ represents hydrogen or halo, and in particular fluoro.

In a third more preferred embodiment the triaryl derivative of the invention is a compound represented by Formula I, IA, IB, IC or ID, wherein

R¹ represents hydrogen;

R² represents hydroxy or alkoxy, and in particular methoxy;

R³ represents halo, and in particular fluoro; and

R⁴ represents trifluoromethyl, trifluoromethoxy or cyano.

In a fourth more preferred embodiment the triaryl derivative of the invention is a compound represented by Formula I, IA, IB, IC or ID, wherein

R¹ represents hydrogen;

R² represents sulfamoyl;

R³ represents halo, and in particular fluoro;

R⁴ represents trifluoromethyl; and

R⁵ represents hydrogen or halo, and in particular fluoro.

In a fifth more preferred embodiment the triaryl derivative of the invention is a compound represented by Formula I, IA, IB, IC or ID, wherein

R¹ represents hydrogen;

R² represents hydroxy or alkoxy, and in particular methoxy;

R³ represents halo, and in particular fluoro; and

R⁴ represents trifluoromethyl.

In a sixth more preferred embodiment the triaryl derivative of the invention is a compound represented by Formula I, IA, IB, IC or ID, wherein

R¹ represents hydrogen;

R² represents hydroxy or alkoxy, and in particular methoxy; and both of R³ and R⁴ represent halo, trifluoromethyl, trifluoromethoxy or cyano.

In a seventh more preferred embodiment the triaryl derivative of the invention is a compound represented by Formula I, IA, IB, IC or ID, wherein

R¹ represents hydrogen;

R² represents hydroxy or alkoxy, and in particular methoxy; and

both of R³ and R⁴ represent halo or trifluoromethyl.

In an eight more preferred embodiment the triaryl derivative of the invention is a compound represented by Formula I, IA, IB, IC or ID, wherein

R¹ represents hydrogen;

R² represents hydroxy or alkoxy, and in particular methoxy; and

both of R³ and R⁴ represent halo, and in particular fluoro or chloro.

In a most preferred embodiment the triaryl derivative of the invention is
4-(2,4-Dichloro-phenyl)-6-(4-methoxy-phenyl)-pyrimidin-5-ylamine;
4-[5-Amino-6-(2,4-dichloro-phenyl)-pyrimidin-4-yl]-phenol;
4-(2-Fluoro-4-trifluoromethyl-phenyl)-6-(4-methoxy-phenyl)-pyrimidin-5-ylamine;
4-[5-Amino-6-(2-fluoro-4-trifluoromethyl-phenyl)-pyrimidin-4-yl]-phenol;
5′,2″-Difluoro-4-methoxy-4″-trifluoromethyl-[1,1′;3′,1″]terphenyl-2′-ylamine;
2′-Amino-5′,2″-difluoro-4″-trifluoromethyl-[1,1′;3′,1″]terphenyl-4-ol;
4-(2,4-Dimethoxy-phenyl)-6-(2-fluoro-4-trifluoromethyl-phenyl)-pyrimidin-5-ylamine;
4-[5-Amino-6-(2-fluoro-4-trifluoromethyl-phenyl)-pyrimidin-4-yl]-benzene-1,3-diol;
2-[5-Amino-6-(2-fluoro-4-trifluoromethyl-phenyl)-pyrimidin-4-yl]-5-methoxy-phenol;
4-(2-Fluoro-4-trifluoromethyl-phenyl)-6-(1H-indol-5-yl)-pyrimidin-5-ylamine;
4-[5-Amino-6-(2-fluoro-4-trifluoromethyl-phenyl)-pyrimidin-4-yl]-benzenesulfonamide;
4-(3-Chloro-4-methoxy-phenyl)-6-(2-fluoro-4-trifluoromethyl-phenyl)-2-methyl-pyrimidin-5-ylamine;
4-(2-Fluoro-4-trifluoromethyl-phenyl)-6-(4-methoxy-phenyl)-2-methyl-5-nitro-pyrimidine;
4-Benzo[1,3]dioxol-5-yl-6-(2-fluoro-4-trifluoromethyl-phenyl)-2-methyl-pyrimidin-5-ylamine;
4-(2-Fluoro-4-trifluoromethyl-phenyl)-6-(4-methoxy-phenyl)-2-methyl-pyrimidin-5-ylamine;
4-[5-Amino-6-(2-fluoro-4-trifluoromethyl-phenyl)-2-methyl-pyrimidin-4-yl]-phenol;
4-(2-Fluoro-4-trifluoromethyl-phenyl)-6-(4-methoxy-phenyl)-pyrimidine-2,5-diamine;
4-[2,5-Diamino-6-(2-fluoro-4-trifluoromethyl-phenyl)-pyrimidin-4-yl]-phenol;
3-(2-Fluoro-4-trifluoromethyl-phenyl)-5-(4-methoxy-phenyl)-pyridazin-4-ylamine;
4-[5-Amino-6-(2-fluoro-4-trifluoromethyl-phenyl)-pyridazin-4-yl]-phenol;
3-(2-Fluoro-4-trifluoromethyl-phenyl)-5-(4-methoxy-phenyl)-pyridin-4-ylamine;
4-[4-Amino-5-(2-fluoro-4-trifluoromethyl-phenyl)-pyridin-3-yl]-phenol;
3-(2-Fluoro-4-trifluoromethyl-phenyl)-5-(1H-indol-5-yl)-pyridin-4-ylamine;
5,2′-Difluoro-3-(1H-indol-5-yl)-4′-trifluoromethyl-biphenyl-2-ylamine; or
2′-Amino-5′,2″-difluoro-4″-trifluoromethyl-[1,1′;3′,1″]terphenyl-4-sulfonic acid amide;

a stereoisomer thereof or a mixture of its stereoisomers, or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O—” group, wherein alkyl is as defined above. Examples of preferred alkoxy groups of the invention include methoxy and ethoxy.

### Steric Isomers

It will be appreciated by those skilled in the art that the compounds of the present invention may exist in different stereoisomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid—by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Pharmaceutically Acceptable Salts

The triaryl derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a triaryl derivative of the invention include alkali metal salts, such as the sodium salt of a compound of the invention containing a carboxy group.

### Steric Isomers

It will be appreciated by those skilled in the art that the triaryl derivatives of the present invention may exist in different stereoisomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981). Optical active compounds can also be prepared from optically active starting materials or intermediates.

### Methods of Producing Triaryl Derivatives

The triaryl derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The present invention is devoted to the provision novel modulators of the nicotinic receptors, which modulators are useful for the treatment of diseases or disorders related to the cholinergic receptors, and in particular the nicotinic acetylcholine receptor (nAChR). Preferred compounds of the invention show activity as positive modulators of the nicotinic acetylcholine α7 receptor subtype.

Due to their pharmacological profile the compounds of the invention may be useful for the treatment of diseases or disorders as diverse as those related to the cholinergic system of the central nervous system (CNS), the peripheral nervous system (PNS), diseases or disorders related to smooth muscle contraction, endocrine diseases or disorders, diseases or disorders related to neuro-degeneration, diseases or disorders related to inflammation, pain, and withdrawal symptoms caused by the termination of abuse of chemical substances.

The compounds of the invention may also be useful as diagnostic tools or monitoring agents in various diagnostic methods, and in particular for in vivo receptor imaging (neuroimaging), and they may be used in labelled or unlabelled form.

In a preferred embodiment the disease, disorder or condition contemplated according to the invention, and responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, epilepsy, post-traumatic syndrome, social phobia, a sleeping disorder, pseudodementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to post therapeutic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with drawal symptoms caused by termination of use of addictive substances, including nicotine withdrawal symptoms, opioid withdrawal symptoms including heroin, cocaine and morphine, benzodiazepine withdrawal symptoms including benzodiazepine-like drugs and alcohol.

In a more preferred embodiment the disease, disorder or condition responsive to modulation of nicotinic acetylcholine receptors is a cognitive disorder, psychosis, schizophrenia or depression.

In another more preferred embodiment the disease, disorder or condition responsive to modulation of nicotinic acetylcholine receptors is associated with smooth muscle contractions, including convulsive disorders, angina pectoris, premature labour, convulsions, diarrhoea, asthma, epilepsy, tardive dyskinesia, hyperkinesia, premature ejaculation and erectile difficulty.

In still another more preferred embodiment the disease, disorder or condition responsive to modulation of nicotinic acetylcholine receptors is related to the endocrine system, such as thyrotoxicosis and pheochromocytoma.

In yet another more preferred embodiment the disease, disorder or condition responsive to modulation of nicotinic acetylcholine receptors is a neurodegenerative disorder including transient anoxia and induced neuro-degeneration.

In a further more preferred embodiment the disease, disorder or condition responsive to modulation of nicotinic acetylcholine receptors is pain, including mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to post therapeutic neuralgia, or to peripheral nerve injury.

In a further more preferred embodiment the disease, disorder or condition responsive to modulation of nicotinic acetylcholine receptors is an inflammatory skin disorder such as acne and rosacea, Crohn's disease, inflammatory bowel disease, ulcerative colitis, and diarrhoea.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

### Pharmaceutical Compositions

In another aspect the invention provides novel pharmaceutical compositions comprising a therapeutically effective amount of a triaryl derivative of the invention.

While a triaryl derivative of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the triaryl derivative of the invention, or a pharmaceutically acceptable salt or derivative thereof, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in drage, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

The triaryl derivatives of the present invention are valuable nicotinic receptor modulators, and therefore useful for the treatment of a range of ailments involving cholinergic dysfunction as well as a range of disorders responsive to the action of nAChR modulators.

In another aspect the invention provides a method for the treatment, prevention or alleviation of a disease or a disorder or a condition of a living animal body, including a human, which disease, disorder or condition is responsive to modulation of cholinergic receptors, and which method comprises administering to such a living animal body, including a human, in need thereof an effective amount of a triaryl derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### BRIEF DESCRIPTION OF THE DRAWINGS

The present invention is further illustrated by reference to the accompanying drawing, in which FIGS. 1A and 1B show the modulatory effect of Compound 2 (i.e. 4-[5-Amino-6-(2,4-dichloro-phenyl)-pyrimidin-4-yl]-phenol), and FIGS. 2A and 2B show the modulatory effect of Compound 6 (i.e. 2′-Amino-5′,2″-difluoro-4″-trifluoromethyl-[1,1′;3′,1″]terphenyl-4-ol) on acetylcholine currents induced in nAChR α7 receptors expressed in Xenopus oocytes:

FIG. 1A shows current traces induced by 100 μM acetylcholine in the absence and in the presence of 0.01 to 31.6 μM of Compound 2;

FIG. 1B shows the concentration-response relationship for the positive modulation of 100 μM acetylcholine responses induced by Compound 2; i.e. % modulation of control vs. log [c] (M). The calculated EC₅₀-value is 1.2 μM and the maximal modulation of the acetylcholine response is 299%.

FIG. 2A shows current traces induced by 100 μM acetylcholine in the absence and in the presence of 0.01 to 31.6 μM of Compound 6;

FIG. 2B shows the concentration-response relationship for the positive modulation of 100 μM acetylcholine responses induced by Compound 6; i.e. % modulation of control vs. log [c] (M). The calculated EC₅₀-value is 1.4 μM and the maximal modulation of the acetylcholine response is 361%.

### EXAMPLES

The invention is further illustrated with reference to the following examples, which are not intended to be in any way limiting to the scope of the invention as claimed.

### ABBREVIATIONS

DME: 1,2-dimethoxyethane
AcOEt: ethyl acetate
PE: petroleum ether, boiling range 40-60° C.
DCM: anhydrous dichloromethane
CFM: chloroform

### Example 1

### Preparatory Examples

### Preparation of Intermediates

### 4-Chloro-6-(4-methoxy-phenyl)-pyrimidin-5-ylamine (Intermediate Compound 1)

To a solution of commercially available 5-amino-4,6-dichloropyrimidine (1.500 g, 9.1466 mmol) in DME (40 ml) and water (20 ml), 4-methoxy phenyl boronic acid (1.529 g, 10.0613 mmol) and sodium carbonate (1.939 g, 18.2932 mmol) were added. The reaction mixture was degassed and kept under nitrogen atmosphere during the entire course of the reaction. Palladium (II) (bistriphenylphosphine)dichloride (0.321 g, 0.4573 mmol) was added and the resulting reaction mixture, refluxed for 5 hrs, was worked up by addition of water and extraction with AcOEt. The organic phase, dried over anhydrous MgSO4, afforded upon evaporation a dark brown gummy material (2.140 g), which eluted with 20% AcOEt in PE gave 1.050 g (−49% yield) of pure title compound.

### 3-Bromo-5-fluoro-4′-methoxy-biphenyl-2-ylamine (Intermediate compound 2)

To a solution of commercially available 2,6-dibromo-4-fluoroaniline (1.000 g, 3.7187 mmol) in DME (30 ml) and water (10 ml), 4-methoxy phenyl boronic acid (0.565 g, 3.7187 mmol) and sodium carbonate (0.788 g, 7.4374 mmol) were added. The reaction mixture was degassed and kept under nitrogen atmosphere during the entire course of the reaction. Palladium (II) (bistriphenylphosphine)dichloride (0.130 g, 0.1859 mmol) was added and the resulting reaction mixture, refluxed for 2 hrs, was worked up by addition of water and extraction with AcOEt. The organic phase, dried over anhydrous MgSO4, afforded upon evaporation a dark brown gummy material (1.025 g), which eluted with 7% AcOEt in PE gave 0.420 g (−38% yield) of pure title compound.

### 4-Chloro-6-(2-fluoro-4-trifluoromethyl-phenyl)-pyrimidin-5-ylamine (Intermediate Compound 3)

To a solution of commercially available 5-amino-4,6-dichloropyrimidine (4.000 g, 24.391 mmol) in DME (40 ml) and water (20 ml), 2-fluoro-4-(trifluoromethyl)phenylboronic acid (5.071 g, 24.391 mmol) and sodium carbonate (5.170 g, 48.782 mmol) were added. The reaction mixture was degassed and kept under nitrogen atmosphere during the entire course of the reaction. Palladium (II) (bistriphenylphosphine)dichloride (0.856 g, 1.2196 mmol) was added and the resulting reaction mixture, refluxed for 4 hrs, was worked up by addition of water and extraction with AcOEt. The organic phase, dried over anhydrous MgSO4, afforded upon evaporation a dark oily residue (˜7 g), which eluted through silica gel with 3% AcOEt in PE gave 2.900 g (˜33% yield) of pure title compound.

### 4-Chloro-6-(2-fluoro-4-trifluoromethyl-phenyl)-2-methyl-pyrimidin-5-ylamine (Intermediate compound 4)

To a solution of commercially available 5-amino-4,6-dichloro-2-methylpyrimidine (2.500 g, 15.2444 mmol) in DME (100 ml) and water (20 ml), 2-fluoro-4-(trifluoromethyl)phenylboronic acid (3.1696 g, 15.2444 mmol) and sodium carbonate (4.847 g, 45.7332 mmol) were added. The reaction mixture was degassed and kept under nitrogen atmosphere during the entire course of the reaction. Palladium (II) (bistriphenylphosphine)dichloride (0.535 g, 0.7622 mmol) was added and the resulting reaction mixture, heated at 90° C. for 8 hrs, was worked up by addition of water and extraction with AcOEt. The organic phase, dried over anhydrous MgSO4, afforded upon evaporation a dark oily residue (˜4.5 g), which was purified by preparative HPLC, to afford 1.210 g (˜26% yield) of the pure title compound.

### 4-Chloro-6-(4-methoxy-phenyl)-2-methyl-5-nitro-pyrimidine (Intermediate Compound 5)

To a solution of commercially available 4,6-dichloro-2-methyl-5-nitro-pyrimidine (4.000 g, 19.2304 mmol) in dioxane (40 ml), 4-methoxy phenyl boronic acid (2.338 g, 15.3843 mmol) and potassium carbonate (7.973 g, 57.6912 mmol) were added. The reaction mixture was degassed and kept under nitrogen atmosphere during the entire course of the reaction. Palladium (II) (bistriphenylphosphine)dichloride (0.675 g, 0.9615 mmol) was added and the resulting reaction mixture, heated at 90° C. overnight, was worked up by addition of water and extraction with AcOEt. The organic phase, dried over anhydrous MgSO4, afforded upon evaporation a brown oily residue (5.370 g), which eluted through silica gel with 14-16% AcOEt in hexane gave 1.490 g (˜28% yield) of the pure title compound as a yellow solid.

### 4-Chloro-6-(4-methoxy-phenyl)-2-methyl-pyrimidin-5-ylamine (Intermediate Compound

To a solution of commercially available 5-amino-4,6-dichloro-2-methylpyrimidine (6.000 g, 33.7038 mmol) in DME (50 ml) and water (10 ml), 4-methoxy phenyl boronic acid (5.122 g, 33.7038 mmol) and sodium carbonate (7.144 g, 67.4076 mmol) were added. The reaction mixture was degassed and kept under nitrogen atmosphere during the entire course of the reaction. Palladium (II) (bistriphenylphosphine)dichloride (1.183 g, 1.6852 mmol) was added and the resulting reaction mixture, heated at 90° C. for 3 hours, was worked up by addition of water and extraction with AcOEt. The organic phase, dried over anhydrous MgSO4, afforded upon evaporation a residue (˜6 g), which eluted through silica gel with 4% AcOEt in hexane gave 4.503 g (˜75% yield) of the pure title compound as a yellow solid.

### 4-Chloro-6-(4-methoxy-phenyl)-pyrimidine-2,5-diamine (Intermediate compound 7)

To a solution of commercially available 4,6-dichloro-pyrimidine-2,5-diamine (2.500 g, 13.9657 mmol) in DME (20 ml) and water (10 ml), 4-methoxy phenyl boronic acid (2.122 g, 13.9657 mmol) and sodium carbonate (2.960 g, 27.9314 mmol) were added. The reaction mixture was degassed and kept under nitrogen atmosphere during the entire course of the reaction. Palladium (II) (bistriphenylphosphine)dichloride (0.490 g, 0.6983 mmol) was added and the resulting reaction mixture, heated at 90° C. for 2 hrs, was worked up by addition of water and extraction with AcOEt. The organic phase, dried over anhydrous MgSO4, afforded upon evaporation a dark solid residue (˜3.5 g), which eluted through silica gel with 15% AcOEt in hexane gave 2.501 g (˜60% yield) of the pure title compound as a yellow solid.

### 5-Chloro-3-(2-fluoro-4-trifluoromethyl-phenyl)-pyridazin-4-ylamine (Intermediate compound 8)

To a solution of 3,5-dichloro-pyridazin-4-ylamine (2.000 g, 12.1955 mmol) prepared as described by Kelley et al. (Kelley J L, Thompson J B, Styles V L, Soroko F E, Cooper B R: Synthesis and anticonvulsant of 3H-imidazo[4,5-c]pyridazine, 1H-imidazo[4,5-d]pyridazine and 1H-benzimidazole analog of 9-(2-fluorobenzyl)-6-methylamino-9H-purine; Journal of Heterocyclic Chemistry 1995 32 1423] in dioxane (100 ml) and water (50 ml), 2-fluoro-4-(trifluoromethyl)phenylboronic acid (8.241 g, 39.6354 mmol) and sodium carbonate (3.231 g, 30.4887 mmol) were added. The reaction mixture was degassed and kept under nitrogen atmosphere during the entire course of the reaction. 1,1′-Bis(diphenylphosphino)ferrocene palladium dichloride (0.625 g, 0.8537 mmol) was added and the resulting reaction mixture, heated at 100° C. for 2 hrs, was worked up by addition of water and extraction with CFM. The organic phase, dried over anhydrous MgSO4, afforded upon evaporation a brown gummy residue (˜3.3 g), which eluted through silica gel with 15% AcOEt in hexane gave 2.202 g (˜67% yield) of the pure title compound as a white solid.

### 3-Bromo-5-(4-methoxy-phenyl)-pyridin-4-ylamine (Intermediate compound 9)

To a solution of commercially available 4-amino-3,5-dibromopyridine (1.500 g, 5.9545 mmol) in DME (40 ml) and water (20 ml), 4-methoxy phenyl boronic acid (0.995 g, 6.55 mmol) and sodium carbonate (1.262 g, 11.909 mmol) were added. The reaction mixture was degassed and kept under nitrogen atmosphere during the entire course of the reaction. Palladium (II) (bistriphenylphosphine)dichloride (0.209 g, 0.2977 mmol) was added and the resulting reaction mixture, heated at 90° C. for 4 hours, was worked up by addition of water and extraction with AcOEt. The organic phase, dried over anhydrous MgSO4, afforded upon evaporation a yellow gummy residue (1.650 g), which eluted through silica gel with 20% AcOEt in hexane gave 1.500 g (˜54% yield) of the pure title compound as a white solid.

### 3-Bromo-5-(2-fluoro-4-trifluoromethyl-phenyl)-pyridin-4-ylamine (Intermediate Compound 10)

To a solution of commercially available 4-amino-3,5-dibromopyridine (1.000 g, 3.9697 mmol) in DME (25 ml) and water (12 ml), 2-fluoro-4-(trifluoromethyl)phenylboronic acid (0.908 g, 4.3667 mmol) and sodium carbonate (0.841 g, 7.9394 mmol) were added. The reaction mixture was degassed and kept under nitrogen atmosphere during the entire course of the reaction. Palladium (II) (bistriphenylphosphine)dichloride (0.139 g, 0.1985 mmol) was added and the resulting reaction mixture, heated at 90° C. for 2 hours, was worked up by addition of water and extraction with AcOEt. The organic phase, dried over anhydrous MgSO4, afforded upon evaporation a yellow gummy residue (˜1.3 g), which eluted through silica gel with 15% AcOEt in hexane gave 0.520 g (˜39% yield) of the pure title compound as a white solid.

### 3-Bromo-5,2′-difluoro-4′-trifluoromethyl-biphenyl-2-ylamine (Intermediate Compound 11)

To a solution of commercially available 2,6-dibromo-4-fluoroaniline (2.319 g, 11.1561 mmol) in DME (30 ml) and water (15 ml), 2-fluoro-4-(trifluoromethyl)phenylboronic acid (3.000 g, 11.1561 mmol) and sodium carbonate (2.956 g, 27.8902 mmol) were added. The reaction mixture was degassed and kept under nitrogen atmosphere during the entire course of the reaction. Palladium (II) (bistriphenylphosphine)dichloride (0.392 g, 0.5578 mmol) was added and the resulting reaction mixture, heated at 90° C. for 8 hours, was worked up by addition of water and extraction with AcOEt. The organic phase, dried over anhydrous MgSO4, afforded upon evaporation a yellow gummy residue (3.920 g), which eluted through silica gel with 2% AcOEt in hexane gave 1.501 g (˜38% yield) of the pure title compound as a white solid.

### Preparation of Final Compounds

### 4-(2,4-Dichloro-phenyl)-6-(4-methoxy-phenyl)-pyrimidin-5-ylamine (Compound 1)

To a solution of 4-chloro-6-(4-methoxy-phenyl)-pyrimidin-5-ylamine (Intermediate compound 1; 0.900 g, 3.8189 mmol) in DME (30 ml) and water (15 ml), 2,4-dichlorophenylboronic acid (0.802 g, 4.2008 mmol) and sodium carbonate (0.8095 g, 7.6378 mmol) were added. The reaction mixture was degassed and kept under nitrogen atmosphere during the entire course of the reaction. Palladium (II) (bistriphenylphosphine)dichloride (0.134 g, 0.1909 mmol) was added and the resulting reaction mixture, refluxed for 3 hrs, was worked up by addition of water and extraction with AcOEt. The organic phase, dried over anhydrous MgSO4, afforded upon evaporation a dark brown gummy material (1.029 g), which eluted with 15% AcOEt in PE gave 0.850 g (˜64% yield) of the pure title compound.

LC-ESI−HRMS of [M+H]+ shows 346.0504 Da. Calc. 346.051393 Da, dev. −2.9 ppm. M.p.=141.3-142.1° C.

### 4-[5-Amino-6-(2,4-dichloro-phenyl)-pyrimidin-4-yl]-phenol (Compound 2)

To a solution of 4-(2,4-dichloro-phenyl)-6-(4-methoxy-phenyl)-pyrimidin-5-ylamine (Compound 1; 0.250 g, 0.7221 mmol) in DCM (15 ml), cooled to −78° C. and kept under nitrogen, a solution of boron tribromide (1.270 g, 5.0547 mmol) in DCM (5 ml) was added dropwise. The reaction mixture was allowed to attain room temperature spontaneously and stirred overnight. The mixture was then cooled again in an ice-salt bath and the excess of the reagent was decomposed by treatment with methanol (5 ml) followed by water (15 ml). The organic layer was washed with water, then extracted with 10% NaOH. Acidification of the alkaline extract and subsequent extraction with CFM provided 0.230 g (96% yield) of pure title compound.

M.p.=97.3-98.2° C. LC-ESI−HRMS of [M+H]+ shows 332.0349 Da. Calc. 332.035743 Da, dev. −2.5 ppm.

### 4-(2-Fluoro-4-trifluoromethyl-phenyl)-6-(4-methoxy-phenyl-1)-pyrimidin-5-ylamine (Compound 3)

To a solution of 4-chloro-6-(4-methoxy-phenyl)-pyrimidin-5-ylamine (Intermediate compound 1; 0.900 g, 3.8189 mmol) in DME (50 ml) and water (25 ml), 2-fluoro-4-(trifluoromethyl)phenylboronic acid (0.952 g, 4.5827 mmol) and sodium carbonate (0.809 g, 7.6378 mmol) were added. The reaction mixture was degassed and kept under nitrogen atmosphere during the entire course of the reaction. Palladium (II) (bistriphenylphosphine)dichloride (0.134 g, 0.1909 mmol) was added and the resulting reaction mixture, refluxed for 4 hrs, was worked up by addition of water and extraction with AcOEt. The organic phase, dried over anhydrous MgSO4, afforded upon evaporation a brown material (1.325 g), which eluted with 20% AcOEt in PE gave 0.801 g (˜58% yield) of the pure title compound.

M.p.=133.8-134.9° C. LC-ESI−HRMS of [M+H]+ shows 364.1061 Da. Calc. 364.107299 Da, dev. −3.3 ppm.

### 4-[5-Amino-6-(2-fluoro-4-trifluoromethyl-phenyl-1)-pyrimidin-4-yl]-phenol (Compound 4)

To a solution of 4-(2-fluoro-4-trifluoromethyl-phenyl)-6-(4-methoxy-phenyl)-pyrimidin-5-ylamine (Compound 3; 0.300 g, 0.8257 mmol) in DCM (20 ml), cooled to −78° C. and kept under nitrogen, a solution of boron tribromide (1.450 g, 5.7799 mmol) in DCM (5 ml) was added dropwise. The reaction mixture was allowed to attain room temperature spontaneously and stirred overnight. The mixture was then cooled again in an ice-salt bath and the excess of the reagent was decomposed by treatment with methanol (10 ml) followed by water (15 ml). The organic layer was washed with water, then extracted with 10% NaOH. Acidification of the alkaline extract and subsequent extraction with CFM provided 0.270 g (94% yield) of pure title compound.

M.p.=159.3-160.2° C. LC-ESI−HRMS of [M+H]+ shows 350.0905 Da. Calc. 350.091649 Da, dev. −3.3 ppm.

### 5′,2″-Difluoro-4-methoxy-4″-trifluoromethyl-[1,1′;3′,1″]terphenyl-2′-ylamine (Compound 5)

To a solution of 3-bromo-5-fluoro-4′-methoxy-biphenyl-2-ylamine (Intermediate compound 2; 0.400 g, 1.3507 mmol) in DME (20 ml) and water (10 ml), 2-fluoro-4-(trifluoromethyl)phenylboronic acid (0.3089 g, 1.4858 mmol) and sodium carbonate (0.286 g, 2.7014 mmol) were added. The reaction mixture was degassed and kept under nitrogen atmosphere during the entire course of the reaction. Palladium (II) (bistriphenylphosphine)dichloride (0.047 g, 0.0675 mmol) was added and the resulting reaction mixture, refluxed for 3 hrs, was worked up by addition of water and extraction with AcOEt. The organic phase, dried over anhydrous MgSO4, afforded upon evaporation a dark brown material (0.501 g), which eluted with 3% AcOEt in PE gave 0.365 g (˜71% yield) of the pure title compound.

M.p.=85.2-86.3° C. LC-ESI−HRMS of [M+H]+ shows 380.1055 Da. Calc. 380.107379 Da, dev. −4.9 ppm.

### 2′-Amino-5′,2″-difluoro-4″-trifluoromethyl-[1,1′;3′,1″]terphenyl-4-ol (Compound 6)

To a solution of 5′,2″-difluoro-4-methoxy-4″-trifluoromethyl-[1,1′;′,1″]terphenyl-2′-ylamine (Compound 5; 0.200 g, 0.5273 mmol) in DCM (15 ml), cooled to −78° C. and kept under nitrogen, a solution of boron tribromide (0.925 g, 3.6911 mmol) in DCM (5 ml) was added dropwise. The reaction mixture was allowed to attain room temperature spontaneously and stirred overnight. The mixture was then cooled again in an ice-salt bath and the excess of the reagent was decomposed by treatment with methanol (5 ml) followed by water (15 ml). The organic layer was washed with water, then extracted with 10% NaOH. Acidification of the alkaline extract and subsequent extraction with CFM provided 0.185 g of crude compound. This latter was purified by flash chromatography by eluting with 8% AcOEt, to afford 0.135 g (70% yield) of the pure title compound.

M.p.=97.8-98.9° C. LC-ESI−HRMS of [M+H]+ shows 366.0908 Da. Calc. 366.091729 Da, dev. −2.5 ppm.

### 4-(2,4-Dimethoxy-phenyl)-6-(2-fluoro-4-trifluoromethyl-phenyl)-pyrimidin-5-ylamine (Compound 7)

The compound was prepared upon reaction of Intermediate Compound 3 and commercially available 2,4-dimethoxyphenylboronic acid by following the experimental procedure described for Compound 1 (yield 50%, M.p. 191.5-192.8° C.).

### 4-[5-Amino-6-(2-fluoro-4-trifluoromethyl-phenyl)-pyrimidin-4-yl]-benzene-1,3-diol (Compound 8); and

### 2-[5-Amino-6-(2-fluoro-4-trifluoromethyl-phenyl)-pyrimidin-4-yl]-5-methoxy-phenol (Compound 9)

To a solution of 4-(2,4-dimethoxy-phenyl)-6-(2-fluoro-4-trifluoromethyl-phenyl)-pyrimidin-5-ylamine (Compound 7; 1.000 g, 2.5423 mmol) in DCM (20 ml), cooled to −78° C. and kept under nitrogen, a solution of an excess of boron tribromide (7 ml) in DCM (20 ml) was added dropwise. The reaction mixture was allowed to attain room temperature spontaneously and stirred overnight. The mixture was then cooled again in an ice-salt bath and the excess of the reagent was decomposed by treatment with methanol (10 ml) followed by water (20 ml). The organic layer was washed with water, then extracted with 10% NaOH. Acidification of the alkaline extract and subsequent extraction with CFM provided a mixture of the title compounds (0.850 g, 91% yield). These were separated by preparative HPLC, to afford Compound 8 (0.170 g, yield 18%, m.p. 150.0-151.5° C.) and Compound 9 (0.330 g, 34% yield, m.p. 100.0-103.0° C.).

### 4-(2-Fluoro-4-trifluoromethyl-phenyl)-6-(1H-indol-5-yl)-pyrimidin-5-ylamine (Compound 10)

The compound was prepared upon reaction of Intermediate compound 3 and commercially available 5-indolylboronic acid by following the experimental procedure described for Compound 1 (yield 57%, M.p. 216.1-217.8° C., LC-ESI−HRMS of [M+H]+ shows 373.107 Da. Calc. 373.107088 Da, dev. −0.2 ppm).

### 4-[5-Amino-6-(2-fluoro-4-trifluoromethyl-phenyl)-pyrimidin-4-yl]-benzenesulfonamide (Compound 11)

The compound was prepared upon reaction of Intermediate compound 3 and commercially available 4-aminosulfonylphenylboronic acid by following the experimental procedure described for Compound 1 (yield 53%, M.p. 174.8-176.1° C., LC-ESI−HRMS of [M+H]+ shows 413.0686 Da. Calc. 413.068989 Da, dev. −0.9 ppm).

### 4-(3-Chloro-4-methoxy-phenyl)-6-(2-fluoro-4-trifluoromethyl-phenyl)-2-methyl-pyrimidin-5-ylamine (Compound 12)

The compound was prepared upon reaction of Intermediate compound 4 and commercially available 3-chloro-4-methoxyphenylboronic acid by following the experimental procedure described for Compound 1 (yield 62%, M.p. 104.5-105.2° C.).

### 4-(2-Fluoro-4-trifluoromethyl-phenyl)-6-(4-methoxy-phenyl)-2-methyl-5-nitro-pyrimidine (Compound 13)

To a solution of 4-chloro-6-(4-methoxy-phenyl)-2-methyl-5-nitro-pyrimidine (Intermediate compound 5; 1.200 g, 4.2906 mmol) in DME (25 ml), 2-fluoro-4-(trifluoromethyl)phenylboronic acid (1.159 g, 5.5778 mmol) and sodium carbonate (1.364 g, 12.8718 mmol) were added. The reaction mixture was degassed and kept under nitrogen atmosphere during the entire course of the reaction. [1,1′-Bis(diphenylphosphino)ferrocene]dichloropalladium(II), complex with dichloromethane(1:1) (0.175 g, 0.2145 mmol) was added and the resulting reaction mixture, heated at 90° C. overnight, was worked up by addition of water and extraction with AcOEt. The organic phase, dried over anhydrous MgSO4, afforded upon evaporation a brown gummy residue (1.750 g), which eluted through silica gel with 8-10% AcOEt in hexane gave 1.200 g (˜64% yield) of the pure title compound as a yellow solid. M.p. 100.8-101.8° C.

### 4-Benzo[1,3]-dioxol-5-yl-6-(2-fluoro-4-trifluoromethyl-phenyl)-2-methyl-pyrimidin-5-ylamine (Compound 14)

The compound was prepared upon reaction of Intermediate compound 4 and commercially available 3,4-methylenedioxyphenylboronic acid by following the experimental procedure described for Compound 1 (yield 84%, M.p. 162.5-163.3° C.).

### 4-(2-Fluoro-4-trifluoromethyl-phenyl)-6-(4-methoxy-phenyl)-2-methyl-pyrimidin-5-ylamine (Compound 15)

The compound was prepared upon reaction of Intermediate compound 6 and commercially available 2-fluoro-4-(trifluoromethyl)phenylboronic acid by following the experimental procedure described for Compound 1 (yield 52%, M.p. 129.2-130.6° C. LC-ESI−HRMS of [M+H]+ shows 378.1236 Da. Calc. 378.122404 Da, dev. 3.2 ppm).

### 4-[5-Amino-6-(2-fluoro-4-trifluoromethyl-phenyl)-2-methyl-pyrimidin-4-yl]-phenol (Compound 16)

The compound was prepared from 4-(2-fluoro-4-trifluoromethyl-phenyl)-6-(4-methoxy-phenyl)-2-methyl-pyrimidin-5-ylamine (Compound 15) upon demethylation reaction with boron tribromide as described for Compound 2 (yield 32%, M.p. 175.4-176.9° C. LC-ESI−HRMS of [M+H]+ shows 364.107 Da. Calc. 364.106754 Da, dev. 0.7 ppm).

### 4-(2-Fluoro-4-trifluoromethyl-phenyl)-6-(4-methoxy-phenyl)-pyrimidine-2,5-diamine (Compound 17)

The compound was prepared upon reaction of Intermediate compound 7 and commercially available 2-fluoro-4-(trifluoromethyl)phenylboronic acid by following the experimental procedure described for Compound 1 (yield 83%, M.p. 125.8-127.1° C. LC-ESI−HRMS of [M+H]+ shows 379.1188 Da. Calc. 379.117653 Da, dev. 3 ppm).

### 4-[2,5-Diamino-6-(2-fluoro-4-trifluoromethyl-phenyl)-pyrimidin-4-yl]-phenol (Compound 18)

The compound was prepared from 4-(2-Fluoro-4-trifluoromethyl-phenyl)-6-(4-methoxy-phenyl)-pyrimidine-2,5-diamine (Compound 17) upon demethylation reaction with boron tribromide as described for Compound 2 (yield 36%, M.p. 226.3-227.5° C. LC-ESI−HRMS of [M+H]+ shows 365.1022 Da. Calc. 365.102003 Da, dev. 0.5 ppm).

### 3-(2-Fluoro-4-trifluoromethyl-phenyl)-5-(4-methoxy-phenyl)-pyridazin-4-ylamine (Compound 19)

To a solution of 5-chloro-3-(2-fluoro-4-trifluoromethyl-phenyl)-pyridazin-4-ylamine (Intermediate compound 8; 2.000 g, 6.8579 mmol) in dioxane (100 ml) and water (50 ml), 4-methoxy phenyl boronic acid (1.251 g, 8.2295 mmol) and sodium carbonate (1.817 g, 17.1447 mmol) were added. The reaction mixture was degassed and kept under nitrogen atmosphere during the entire course of the reaction. 1,1′-Bis(diphenylphosphino)ferrocene palladium dichloride (0.251 g, 0.3429 mmol) was added and the resulting reaction mixture, heated at 100° C. for 2 hours, was worked up by addition of water and extraction with CFM. The organic phase, dried over anhydrous MgSO4, afforded upon evaporation a brown gummy residue (˜2 g), which eluted through silica gel with 55% AcOEt in hexane gave 1.206 g (˜60% yield) of the pure title compound as an off-white solid. M.p. 215.3-216.2° C.

### 4-[5-Amino-6-(2-fluoro-4-trifluoromethyl-phenyl)-pyridazin-4-yl]-phenol (Compound 20)

The compound was prepared from 3-(2-fluoro-4-trifluoromethyl-phenyl)-5-(4-methoxy-phenyl)-pyridazin-4-ylamine (Compound 19) upon demethylation reaction with boron tribromide as described for Compound 2 (yield 85%, M.p. 251.0-252.2° C.).

### 3-(2-Fluoro-4-trifluoromethyl-phenyl)-5-(4-methoxy-phenyl)-pyridin-4-ylamine (Compound 21)

The compound was prepared upon reaction of Intermediate compound 9 and commercially available 2-fluoro-4-(trifluoromethyl)phenylboronic acid by following the experimental procedure described for Compound 1 (yield 68%, M.p. 169.5-170.3° C., LC-ESI−HRMS of [M+H]+ shows 363.112485 Da. Calc. 363.111505 Da, dev. 2.7 ppm).

### 4-[4-Amino-5-(2-fluoro-4-trifluoromethyl-phenyl)-pyridin-3-yl]-phenol (Compound 22)

The compound was prepared from 3-(2-fluoro-4-trifluoromethyl-phenyl)-5-(4-methoxy-phenyl)-pyridin-4-ylamine (Compound 21) upon demethylation reaction with boron tribromide as described for Compound 2 (yield 41%, M.p. 133.6-134.8° C., LC-ESI−HRMS of [M+H]+ shows 349.0977345 Da. Calc. 349.095855 Da, dev. 5.4 ppm).

### 3-(2-Fluoro-4-trifluoromethyl-phenyl)-5-(1H-indol-5-yl)-pyridin-4-ylamine (Compound 23)

The compound was prepared upon reaction of Intermediate compound 10 and commercially available 5-indolylboronic acid by following the experimental procedure described for Compound 1 (yield 36%, M.p. 103-106.0° C., LC-ESI−HRMS of 20 [M+H]+ shows 372.1124 Da. Calc. 372.111839 Da, dev. 1.5 ppm).

### 5,2′-Difluoro-3-(1H-indol-5-yl)-4′-trifluoromethyl-biphenyl-2-ylamine (Compound 24)

The compound was prepared upon reaction of Intermediate compound 11 and commercially available 5-indolylboronic acid by following the experimental procedure described for Compound 1 (yield 83%, LC-ESI−HRMS of [M+H]+ shows 389.1082 Da. Calc. 389.107168 Da, dev. 2.7 ppm).

### 2′-Amino-5′,2″-difluoro-4″-trifluoromethyl-[1,1′;3′,1″]terphenyl-4-sulfonic acid amide (Compound 25)

The compound was prepared upon reaction of Intermediate compound 11 and commercially available 4-aminosulfonylphenylboronic acid by following the experimental procedure described for Compound 1 (yield 77%, M.p. 189.4-191.2° C., LC-ESI−HRMS of [M+H]+ shows 429.068 Da. Calc. 429.069069 Da, dev. −2.5 ppm).

### Example 2

### Biological Activity

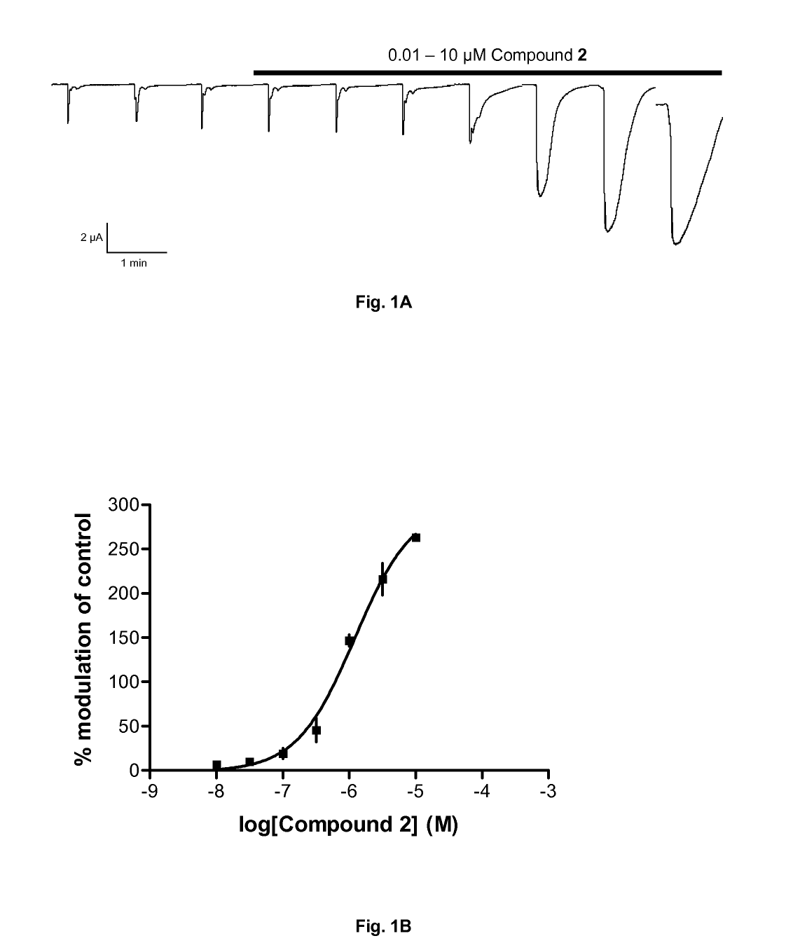

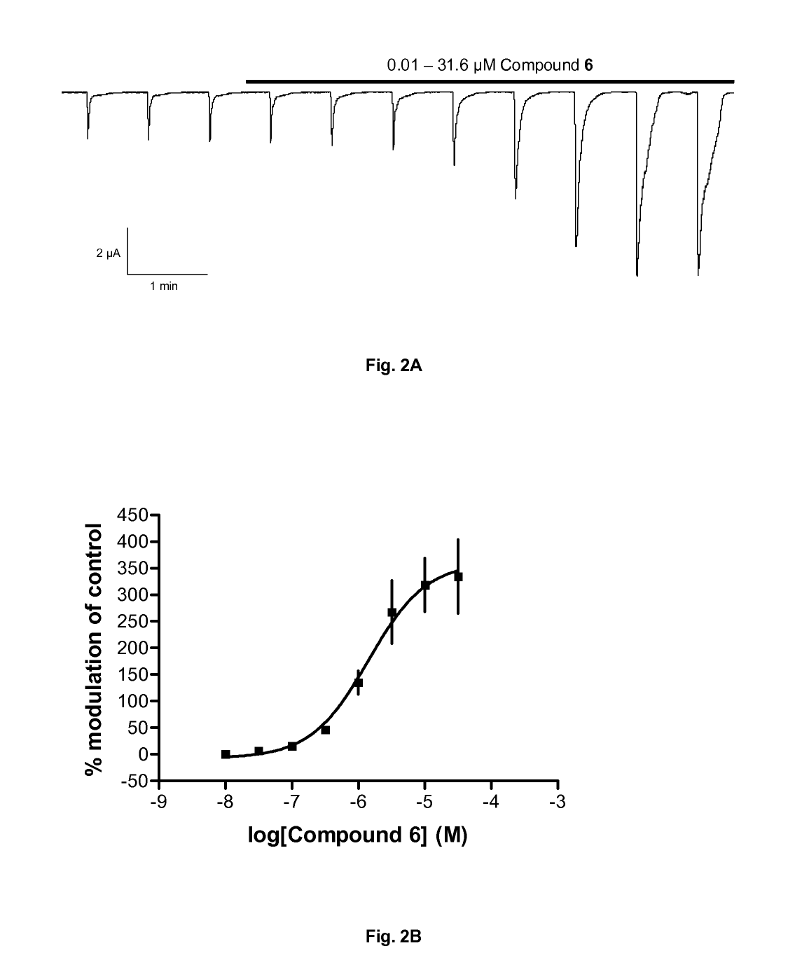

In this example the positive modulation of wild-type nAChR α7 receptors by Compound 2 (i.e. 4-[5-Amino-6-(2,4-dichloro-phenyl)-pyrimidin-4-yl]-phenol; FIGS. 1A and 1B) and by Compound 6 (i.e. 2′-Amino-5′,2″-difluoro-4″-trifluoromethyl-[1,1′;3′,1″]terphenyl-4-ol; FIGS. 2A and 2B) was determined using nAChR α7 receptors heterologously expressed in Xenopus laevis oocytes.

The electrical current through the nAChR α7 channel was measured using conventional two-electrode voltage clamp and nAChR α7 currents were activated by applying pulses of agonist-containing solution onto the nAChR α7 expressing oocyte.

In brief, the oocytes were placed in a recording chambers and continuously superfused with an Oocyte Ringer (OR) solution containing 90 mM NaCl, 2.5 mM KCl, 2.5 mM CaCl₂, 1 mM MgCl₂ and 5 mM HEPES (pH adjusted to 7.4). The oocytes were clamped at −60 mV and currents were induced by applying 20 s pulses of 100 μM acetylcholine dissolved in OR. The intervals between the acetylcholine applications were 5 minutes, during which the oocytes were washed with OR. The first three applications were control applications to insure a constant response level of 100 μM acetylcholine. For the subsequent 8 test applications, increasing concentrations (0.01-31.6 μM) of Compound 2 or Compound 6 was applied 30 s before and during the acetylcholine (100 μM) application, which caused a robust increase in the acetylcholine-induced current amplitude.

The positive modulation in the presence of Compound 2 or Compound 6 was calculated as (test-control)/control*100% and the concentration response curve for this positive modulation was fitted to the sigmoidal logistic equation: I=Iₘₐₓ/(1+(EC₅₀/[compound])ⁿ), where Iₘₐₓ represents the maximal modulation of the control response, EC₅₀ is the concentration causing a half maximal response, and n is the slope coefficient.

The calculated EC₅₀ values for Compounds 2 and 6 were 1.2 μM and 1.4 μM, respectively. The calculated Iₘₐₓ values for Compounds 2 and 6 were 299% and 361%, respectively.

1. A triaryl derivative represented by Formula I 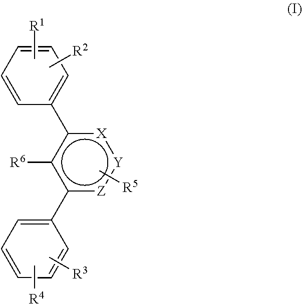
a stereoisomer thereof or a mixture of its stereoisomers, or a pharmaceutically acceptable salt thereof, wherein
all of X, Y and Z represent CH; or
one or two of X, Y and Z represent N; and
the others of X, Y and Z represent CH; and
R¹, R², R³, R⁴ and R⁵, independently of each other, represent hydrogen, halo, trifluoromethyl, trifluoromethoxy, cyano, hydroxyl, alkoxy, alkyl, amino or sulfamoyl; or
R¹ and R², together with the phenyl ring to which they are attached form an indolyl ring or a benzo-dioxolyl ring; and
R³, R⁴ and R⁵ are as defined above; and
R⁶ represents amino or nitro;
provided, however,
that not all of R¹, R², R³, R⁴ and R⁵ represent hydrogen (i.e. at least one of R¹, R², R³, R⁴ and R⁵ is different from hydrogen);
if R⁵ represents halo, methoxy or amino, not all of R¹, R², R³ and R⁴ represent hydrogen;
if all of X, Y and Z represent CH, then one of R¹ and R², or one of R³ and R⁴, do not represent chloro if the other two of R¹, R², R³ and R⁴ represent hydrogen; and
if X and Z represent N, and R⁵ represent hydrogen, then one of R¹ and R², or one of R³ and R⁴, do not represent methoxy if the other two of R¹, R², R³ and R⁴ represent hydrogen.

2. The triaryl derivative of claim 1, a stereoisomer thereof or a mixture of its stereoisomers, or a pharmaceutically acceptable salt thereof, wherein all of X, Y and Z represent CH.

3. The triaryl derivative of claim 1, a stereoisomer thereof or a mixture of its stereoisomers, or a pharmaceutically acceptable salt thereof, wherein
one or two of X, Y and Z represent N; and
the others of X, Y and Z represent CH.

4. The triaryl derivative of any one of claims 1-3, a stereoisomer thereof or a mixture of its stereoisomers, or a pharmaceutically acceptable salt thereof, wherein
R¹, R², R³, R⁴ and R⁵, independently of each other, represent hydrogen, halo, trifluoromethyl, trifluoromethoxy, cyano, hydroxyl, alkoxy, alkyl, amino or sulfamoyl; or
R¹ and R², together with the phenyl ring to which they are attached form an indolyl ring or a benzo-dioxolyl ring; and
R³, R⁴ and R⁵ are as defined above.

5. The triaryl derivative of claim 4, a stereoisomer thereof or a mixture of its stereoisomers, or a pharmaceutically acceptable salt thereof, wherein
R¹ represents hydrogen, halo, hydroxy or alkoxy; and
R² represents hydroxy, alkoxy or sulfamoyl.

6. The triaryl derivative of claim 4, a stereoisomer thereof or a mixture of its stereoisomers, or a pharmaceutically acceptable salt thereof, wherein R¹ and R², together with the phenyl ring to which they are attached form an indolyl ring or a benzo dioxolyl ring.

7. The triaryl derivative of claim 4, a stereoisomer thereof or a mixture of its stereoisomers, or a pharmaceutically acceptable salt thereof, wherein R³ and R⁴, independently of each other, represent halo, trifluoromethyl, trifluoromethoxy or cyano.

8. The triaryl derivative of claim 4, a stereoisomer thereof or a mixture of its stereoisomers, or a pharmaceutically acceptable salt thereof, wherein R⁵ represents hydrogen, halo, trifluoromethyl alkyl or amino.

9. The triaryl derivative of claim 1, a stereoisomer thereof or a mixture of its stereoisomers, or a pharmaceutically acceptable salt thereof, wherein R⁶ represents amino or nitro.

10. The triaryl derivative of claim 1, a stereoisomer thereof or a mixture of its stereoisomers, or a pharmaceutically acceptable salt thereof, wherein
R¹ represents hydrogen;
R² represents hydroxy or alkoxy; and
one of R³ and R⁴ represents halo; and
the other of R³ and R⁴ represents trifluoromethyl, trifluoromethoxy or cyano; or
both of R³ and R⁴ represent halo, trifluoromethyl, trifluoromethoxy or cyano.

11. The triaryl derivative of claim 1, which is
4-(2,4-Dichloro-phenyl)-6-(4-methoxy-phenyl)-pyrimidin-5-ylamine;
4-[5-Amino-6-(2,4-dichloro-phenyl)-pyrimidin-4-yl]-phenol;
4-(2-Fluoro-4-trifluoromethyl-phenyl)-6-(4-methoxy-phenyl)-pyrimidin-5-ylamine;
4-[5-Amino-6-(2-fluoro-4-trifluoromethyl-phenyl)-pyrimidin-4-yl]-phenol;
5′,2″-Difluoro-4-methoxy-4″-trifluoromethyl-[1,1′;3′,1″]terphenyl-2′-ylamine;
2′-Amino-5′,2″-difluoro-4″-trifluoromethyl-[1,1′;3′,1″]terphenyl-4-ol;
4-(2,4-Dimethoxy-phenyl)-6-(2-fluoro-4-trifluoromethyl-phenyl)-pyrimidin-5-ylamine;
4-[5-Amino-6-(2-fluoro-4-trifluoromethyl-phenyl)-pyrimidin-4-yl]-benzene-1,3-diol;
2-[5-Amino-6-(2-fluoro-4-trifluoromethyl-phenyl)-pyrimidin-4-yl]-5-methoxy-phenol;
4-(2-Fluoro-4-trifluoromethyl-phenyl)-6-(1H-indol-5-yl)-pyrimidin-5-ylamine;
4-[5-Amino-6-(2-fluoro-4-trifluoromethyl-phenyl)-pyrimidin-4-yl]-benzenesulfonamide;
4-(3-Chloro-4-methoxy-phenyl)-6-(2-fluoro-4-trifluoromethyl-phenyl)-2-methyl-pyrimidin-5-ylamine;
4-(2-Fluoro-4-trifluoromethyl-phenyl)-6-(4-methoxy-phenyl)-2-methyl-5-nitro-pyrimidine;
4-Benzo[1,3]dioxol-5-yl-6-(2-fluoro-4-trifluoromethyl-phenyl)-2-methyl-pyrimidin-5-ylamine;
4-(2-Fluoro-4-trifluoromethyl-phenyl)-6-(4-methoxy-phenyl)-2-methyl-pyrimidin-5-ylamine;
4-[5-Amino-6-(2-fluoro-4-trifluoromethyl-phenyl)-2-methyl-pyrimidin-4-yl]-phenol;
4-(2-Fluoro-4-trifluoromethyl-phenyl)-6-(4-methoxy-phenyl)-pyrimidine-2,5-diamine;
4-[2,5-Diamino-6-(2-fluoro-4-trifluoromethyl-phenyl)-pyrimidin-4-yl]-phenol;
3-(2-Fluoro-4-trifluoromethyl-phenyl)-5-(4-methoxy-phenyl)-pyridazin-4-ylamine;
4-[5-Amino-6-(2-fluoro-4-trifluoromethyl-phenyl)-pyridazin-4-yl]-phenol;
3-(2-Fluoro-4-trifluoromethyl-phenyl)-5-(4-methoxy-phenyl)-pyridin-4-ylamine;
4-[4-Amino-5-(2-fluoro-4-trifluoromethyl-phenyl)-pyridin-3-yl]-phenol;
3-(2-Fluoro-4-trifluoromethyl-phenyl)-5-(1H-indol-5-yl)-pyridin-4-ylamine;
5,2′-Difluoro-3-(1H-indol-5-yl)-4′-trifluoromethyl-biphenyl-2-ylamine; or
2′-Amino-5′,2″-difluoro-4″-trifluoromethyl-[1,1′;3′,1″]terphenyl-4-sulfonic acid amide;
a stereoisomer thereof or a mixture of its stereoisomers, or a pharmaceutically acceptable salt thereof.

12. A pharmaceutical composition comprising a therapeutically effective amount of a triaryl derivative of claim 1, a stereoisomer thereof or a mixture of its stereoisomers, or a pharmaceutically acceptable addition salt thereof, together with at least one pharmaceutically acceptable carrier or diluent.

13. A triaryl derivative of claim 1, a stereoisomer thereof or a mixture of its stereoisomers, or a pharmaceutically acceptable addition salt thereof, for use as a medicament.

14. (canceled)

15. (canceled)

16. A method of treatment, prevention or alleviation of a disease or a disorder or a condition of a living animal body, including a human, which disorder, disease or condition is responsive to modulation of nicotinic acetylcholine receptors, which method comprises the step of administering to such a living animal body in need thereof a therapeutically effective amount of a triaryl derivative of claim 1, a stereoisomer thereof or a mixture of its stereoisomers, or a pharmaceutically acceptable salt thereof.

17. The method according to claim 16 wherein the disease, disorder or condition responsive to modulation of nicotinic acetylcholine receptors is anxiety, a cognitive disorder, a learning deficit, a memory deficit or dysfunction, Alzheimer's disease, attention deficit, attention deficit hyperactivity disorder, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, periferic neuropathy, autism, dyslexia, tardive dyskinesia, hyperkinesia, epilepsy, post-traumatic syndrome, social phobia, a sleeping disorder, pseudodementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, epilepsy, tardive dyskinesia, hyperkinesia, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with drawal symptoms caused by termination of use of addictive substances, including nicotine withdrawal symptoms, opioid withdrawal symptoms, including heroin, cocaine and morphine, benzodiazepine withdrawal symptoms including benzodiazepine-like drugs and alcohol.
